(19) United States
(12) Patent Application Publication
(10) Pub. No.: US 2023/0377135 A1
(43) Pub. Date: Nov. 23, 2023

(54) SYSTEM, METHOD, AND COMPUTER PROGRAM OF AUTOMATICALLY RECOGNIZING MALOCCLUSION CLASS

(71) Applicant: SHARESMILE BIOTECH CO., LTD., New Taipei City (TW)

(72) Inventors: Kio-Heng SAM, New Taipei City (TW); Cheng-Han YU, New Taipei City (TW)

(21) Appl. No.: 17/750,282

(22) Filed: May 20, 2022

(51) Int. Cl. G06T 7/00; H04N 5/232; G06T 5/00; A61C 19/05

(52) U.S. Cl. CPC G06T 7/0012; H04N 5/23222; H04N 5/232939; G06T 5/00; A61C 19/05; G06T 2207/30036; G06T 2207/30201

(57) ABSTRACT

A system of automatically recognizing a malocclusion class is disclosed. The disclosure is to compute an occlusion grade based on an occlusion image, compute a lower teeth profile grade based on a lower teeth profile image, compute an upper teeth profile grade based on an upper teeth profile image, compute a side face profile grade based on a side face image, and determine a credibility of a malocclusion class based on the grades. A method and a computer program are also disclosed.

### BACKGROUND OF THE DISCLOSURE

### Technical Field

The disclosure relates to a system, a method and a computer program, particularly relates to a system, a method and a computer program of automatically recognizing a malocclusion class.

### Description of Related Art

Malocclusion (or bad bites) indicates that the teeth are misaligned, which is a common condition. The patients with malocclusion may have anxiety of exposing inaesthetic teeth, and malocclusion may further induce oral disease.

In the present medical science, different treatments may be applied to the patients with different malocclusion levels to effectively correct malocclusion.

In the related art, in order to confirm the malocclusion level, the patients need to go to the dental clinic for panoramic x-ray angiography, and the dentist diagnoses the malocclusion level of the patients according to the panoramic x-ray image.

Therefore, the related-art malocclusion diagnostic method has the issue of inconvenience for the patients. In view of this, the inventors have devoted themselves to the aforementioned related art, researched intensively to solve the aforementioned problems.

### SUMMARY OF THE DISCLOSURE

The object of the disclosure is to provide a system, a method and a computer program of automatically recognizing a malocclusion class, which may determine the malocclusion class through the oral image captured by the camera.

In some embodiments, a method of automatically recognizing a malocclusion class is provided. The method includes: a) obtaining, by a computer device, an occlusion image, a lower teeth profile image, an upper teeth profile image, and a side face image; b) computing an occlusion grade based on an occlusion state of a teeth image in the occlusion image; c) computing a lower teeth profile grade based on an arrangement of a lower teeth image in the lower teeth profile image; d) computing an upper teeth profile grade based on an arrangement of an upper teeth image in the lower teeth profile image; e) computing a side face profile grade based on a side face profile in the side face image; f) determining at least one credibility of at least one malocclusion class based on the occlusion grade, the lower teeth profile grade, the upper teeth profile grade, and the side face profile grade; and g) outputting, by the computer device, the malocclusion class with a highest credibility.

In some embodiments, a system of automatically recognizing a malocclusion class is provided. The system includes an occlusion analyzing module, a lower teeth profile analyzing module, an upper teeth profile analyzing module, a side face profile analyzing module, and a credibility computing module. The occlusion analyzing module is configured to compute an occlusion grade based on an occlusion state of a teeth image in an occlusion image of a user. The lower teeth profile analyzing module is configured to compute a lower teeth profile grade based on an arrangement of a lower teeth image in a lower teeth profile image of the user. The upper teeth profile analyzing module is configured to compute an upper teeth profile grade based on an arrangement of an upper teeth image in an upper teeth profile image of the user. The side face profile analyzing module is configured to compute a side face profile grade based on a side face profile in a side face image of the user. The credibility computing module is configured to determine a credibility of at least one malocclusion class based on the occlusion grade, the lower teeth profile grade, the upper teeth profile grade and the side face profile grade, and output the malocclusion class with a highest credibility.

In some embodiments, a computer program of automatically recognizing a malocclusion class is provided. The computer program is configured to be stored in a computer device, and implement the aforementioned method after being executed by the computer device.

The disclosure may be used to automatically recognize the user belonging in which malocclusion class for the reference of orthodontic treatment.

### BRIEF DESCRIPTION OF THE DRAWINGS

The file of this application contains drawings executed in color. Copies of this patent or patent application publication with color drawing(s) will be provided by the Office upon request and payment of the necessary fee. As the color drawings are being filed electronically via EFS-Web, only one set of the drawings is submitted.

FIG. 1 is an architecture diagram of the system of the disclosure in accordance with some embodiments.

FIG. 2 is an architecture diagram of the system of the disclosure in accordance with some embodiments.

FIG. 3 is a schematic diagram of the data stream of the disclosure in accordance with some embodiments.

FIG. 4 is a schematic diagram of calculating credibility of the disclosure in accordance with some embodiments.

FIG. 5 is a flowchart of the method of the disclosure in accordance with some embodiments.

FIG. 6 is a partial flowchart of the method of the disclosure in accordance with some embodiments.

FIG. 7 is a partial flowchart of the method of the disclosure in accordance with some embodiments.

FIG. 8A is a schematic diagram of the occlusion image of the disclosure in accordance with some embodiments.

FIG. 8B is a schematic diagram of the image of the occlusion portion of the disclosure in accordance with some embodiments.

FIG. 9A is a schematic diagram of the lower teeth profile image of the disclosure in accordance with some embodiments.

FIG. 9B is a schematic diagram of the image of the lower teeth portion of the disclosure in accordance with some embodiments.

FIG. 10A is a schematic diagram of the upper teeth profile image of the disclosure in accordance with some embodiments.

FIG. 10B is a schematic diagram of the image of the upper teeth portion of the disclosure in accordance with some embodiments.

FIG. 11A is a schematic diagram of the side face image of the disclosure in accordance with some embodiments.

FIG. 11B is a schematic diagram of the image of the side face portion of the disclosure in accordance with some embodiments.

FIG. 12 is an image schematic diagram of the malocclusion class A1 of the disclosure in accordance with some embodiments.

FIG. 13 is an image schematic diagram of the malocclusion class A2 of the disclosure in accordance with some embodiments.

FIG. 14 is an image schematic diagram of the malocclusion class A3 of the disclosure in accordance with some embodiments.

FIG. 15 is an image schematic diagram of the malocclusion class B of the disclosure in accordance with some embodiments.

### DETAILED DESCRIPTION

The technical contents of this disclosure will become apparent with the detailed description of embodiments accompanied with the illustration of related drawings as follows. It is intended that the embodiments and drawings disclosed herein are to be considered illustrative rather than restrictive.

The disclosure provides a system, a method and a computer program of automatically recognizing a malocclusion class, which may provide the users to inspect their own teeth belonging in which malocclusion class for the reference of whether correction and what orthodontic treatment being needed. As a result, the users may not need to inspect that with professional panoramic x-ray equipment and the assistance from the dentist.

Specifically, the disclosure is used to obtain the oral images of at least four designated angles shot by the user through the camera 11 of the computer device 1, and perform the below-mentioned processing and analyzation to the oral images to calculate the credibility of the malocclusion class in which the teeth profile belongs.

It is worth noting that the oral images captured by users themselves may contain some interferences, such as environmental light, shooting angle, focal length, etc., and the interferences may be regarded as noise during processing and analyzation and influence accuracy of recognition result.

Regarding that, the disclosure does not directly generate binary recognition result (that is, only output “yes” or “no” for determining malocclusion class), but compute and provide the credibility of the malocclusion class (that is, the possibility of belonging in the malocclusion class, the interferences may lower the credibility) to avoid one-time error, which may make the users search inappropriate orthodontic treatment or delay the treatment.

For example, when the specific malocclusion class is with high credibility, the users may confidentially believe that they have malocclusion and immediately search treatment with respect to the malocclusion class. Further, when any one of the malocclusion classes is general credibility (generally, the oral image being provided has the interference), the users may use different images to re-execute the disclosure to exclude the interferences. Moreover, when any one of the malocclusion classes is low credibility, the users may confidentially believe that they do not belong to the malocclusion class.

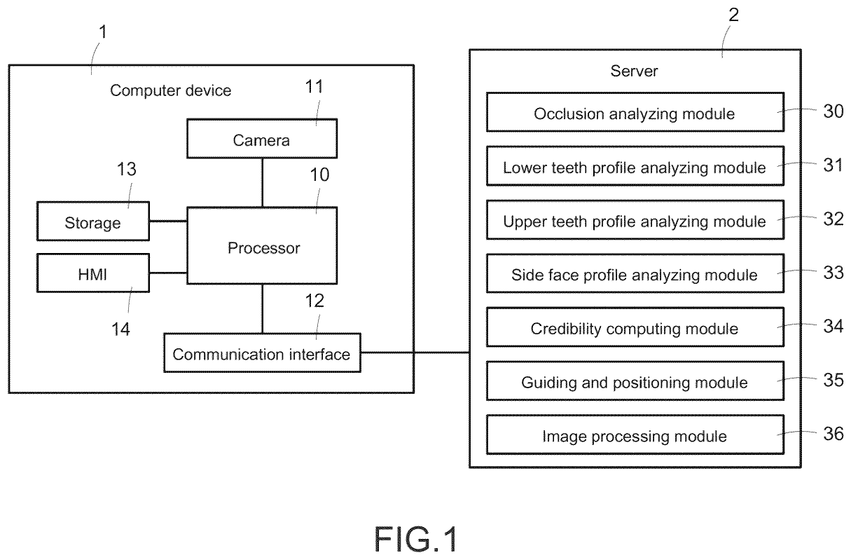

Please refer to FIG. 1, which is an architecture diagram of the system of the disclosure in accordance with some embodiments.

In some embodiments, the disclosure is implemented though cloud computing.

Specifically, the computer device 1, such as the smart phone, laptop, tablet computer, or the other home computers owned by the user, may include a camera 11, a communication interface 12, a storage 13, a human-machine interface (HMI) 14, and a processor 10 electrically connected with all of the aforementioned elements.

The camera 11 may be, for example, a visible light camera used for shooting visible light image.

The communication interface 12 may include a wireless network module (such as Wi-Fi module, Bluetooth module, hive network module, etc.), or a wired network module (such as, an ethernet module, a power line network module, a fiber-optic network module, etc.), or an arbitrary combination of the aforementioned network modules.

The storage 13 may include a volatile storage medium or/and a non-volatile storage medium, such as RAM, ROM, flash memory, hard disk, and/or EEPROM, etc.

The HMI 14 may include an input device (such as, a touch pad, a keyboard, a mouse, etc.) and an output device (such as, a display, a speaker, etc.).

The processor 10 may include MCU, CPU, FPGA, SoC, and/or the other processing circuit module.

In some embodiments, the user may control the computer device 1 to use the camera to shoot the oral image (such as, the after-mentioned occlusion image, lower teeth profile image, upper teeth profile image, and side face image), and connect to the server 2 by the browser or application program through the network (such as, the Internet) to upload the oral image to the server 2.

The server 2 may include an occlusion analyzing module 30, a lower teeth profile analyzing module 31, an upper teeth profile analyzing module 32, a side face profile analyzing module 33, a credibility computing module 34, a guiding and positioning module 35, and/or an image processing module 36.

The server 2 is configured to perform processing and analyzation through the modules 30 to 36 to the oral image obtained from the computer device 1, determine the credibility of the user's teeth profile belonging in each malocclusion class, and output the recognition result to the computer device 1.

The recognition result may be all of the determined malocclusion classes and the credibility, or the malocclusion class with the highest credibility, here is not intended to be limiting.

Therefore, the user may obtain the teeth profile recognition result by the computer device 1 with less powerful but sufficient computing power.

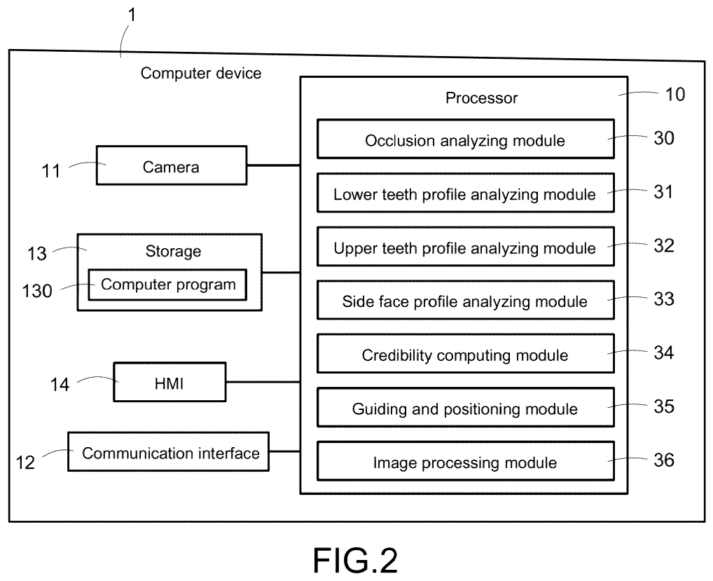

Please refer to FIG. 2, which is an architecture diagram of the system of the disclosure in accordance with some embodiments.

In some embodiments, the disclosure is implemented through dew computing (or ground computing).

The processor 10 may include an occlusion analyzing module 30, a lower teeth profile analyzing module 31, an upper teeth profile analyzing module 32, a side face profile analyzing module 33, a credibility computing module 34, a guiding and positioning module 35, and/or an image processing module 36.

In some embodiments, the computer device 1 may be used to shoot the oral image through the camera 11, perform processing and analyzation through the processor 10 to the oral image to determine the credibility of the user's teeth profile belonging in each malocclusion class, and output the recognition result through the display of the HMI 14.

Therefore, the user may obtain the teeth profile recognition result from the computer device 1 without connecting to the Internet.

It is worth noting that the modules 30 to 36 are connected to each other (electrical connection or informational connection), and are hardware modules (for example, electric circuit module, integrated circuit module, SoC, etc.), or software modules (for example, firmware, operating system, or application program), or a combination of software and hardware, here is not intended to be limiting.

It is worth noting that when the modules 30 to 36 are software modules (for example, application program), the storage 13 may include a non-transitory computer readable record medium (not shown in figures). The non-transitory computer readable record medium is configured to store the computer program 130 of automatically recognizing the malocclusion class. The computer program 130 is configured to record the computer executable code. When the computer device (such as the processor 10 or server 2) is configured to execute the code, the functions corresponding to the modules 30 to 36 are practically implemented.

In some embodiments, when the computer program 130 is executed by the computer device, the functions corresponding to the modules 30 to 36 are practically implemented to complete the method of automatically recognizing the malocclusion class in the after-mentioned embodiments.

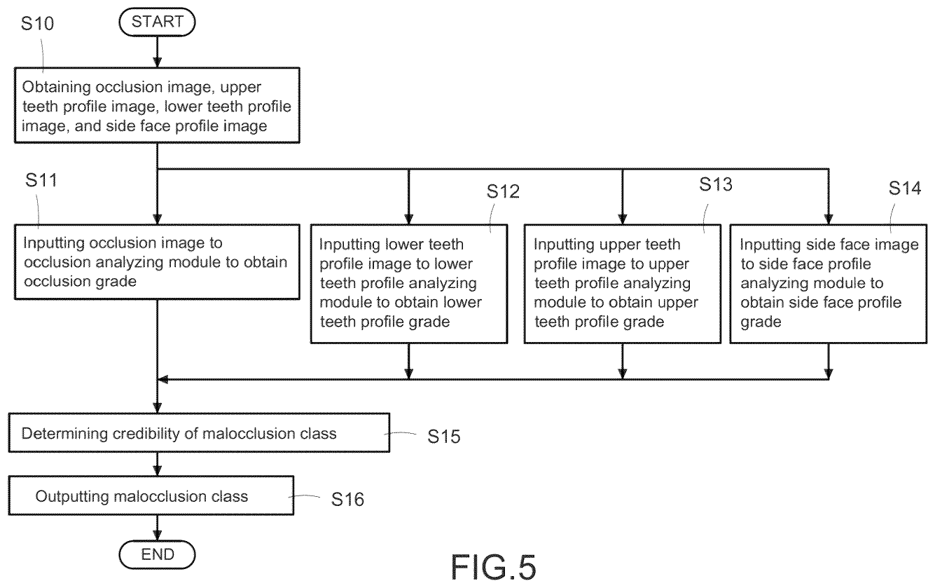

Please refer to FIG. 5, which is a flowchart of the method of the disclosure in accordance with some embodiments. The method of automatically recognizing the malocclusion class in the disclosure includes the steps S10 to S16.

In the step S10, the computer device 1 is controlled by the user to shoot the user's teeth-exposed occlusion, upper teeth profile, lower teeth profile and side face through the camera 11 to obtain the user's occlusion image, lower teeth profile image, upper teeth profile image, and side face image.

In the step S11, the occlusion image is input to the occlusion analyzing module 30. The occlusion analyzing module 30 may be configured to calculate the occlusion grade based on the occlusion state of the teeth image in the occlusion image.

In the step S12, the lower teeth profile image is input to the lower teeth profile analyzing module 31. The lower teeth profile analyzing module 31 may be configured to calculate the lower teeth profile grade based on the arrangement of the lower teeth image in the lower teeth profile image.

In the step S13, the upper teeth profile image is input to the upper teeth profile analyzing module 32. The upper teeth profile analyzing module 32 may be configured to calculate the upper teeth profile grade based on the arrangement of the upper teeth image in the upper teeth profile image.

In the step S14, the side face image is input to the side face profile analyzing module 33. The side face profile analyzing module 33 may be configured to calculate the side face profile grade based on the side face profile in the side face image.

In some embodiments, the occlusion analyzing module 30, the lower teeth profile analyzing module 31 and the upper teeth profile analyzing module 32 may be configured to respectively execute the edge detection to the input images (that is, the occlusion image, lower teeth profile image, and upper teeth profile image) to determine range of the teeth, and determine name of each teeth (such as, first molar on the lower jaw, first molar on the upper jaw, lower incisor, upper incisor, etc.) according to the relative positions of the teeth, and calculate the offsets between any two of the teeth to determine the grade (that is, the occlusion grade, lower teeth profile grade and upper teeth profile grade) based on the offsets.

In some embodiments, the side face profile analyzing module 33 may be configured to execute the edge detection to the input side face image to determine side face profile, and calculate the offsets according to the undulation of the side face profile to determine the grade (that is, the side face profile grade) based on the offsets.

It is worth noting that when the malocclusion is severer, the side face profile may be clearly deformed, and the malocclusion may be determined through the side face image.

In some embodiments, when the offsets between the teeth and the side face are obtained, the rule of Angle's classification may be adopted to determine the probability of the teeth profile in the image belonging in each class of Angle's classification as the aforementioned grades.

In some embodiments, the offset rule may be set differently according to the required correction time of different malocclusion classes.

For example, when the offset between the first molar on the lower jaw and the first molar on the upper jaw is less than a first threshold value, the probability of first malocclusion class may be increased, and when the offset is greater than the first threshold value and less than a second threshold value, the probability of second malocclusion class may be increased. When the offset between the lower incisor and the upper incisor is less than a third threshold value, the probability of first malocclusion class may be increased, and when the offset is greater than the third threshold value, the probability of third malocclusion class may be increased. When the oral protrusion (offset) of the side face is greater than a fourth threshold value, the probability of fourth malocclusion class may be increased. Here is not intended to be limiting.

In some embodiments, the occlusion analyzing module 30, the lower teeth profile analyzing module 31, the upper teeth profile analyzing module 32 and the side face profile analyzing module 33 may include a machine learning model corresponding to the image types, and is configured to execute grading for the input image through the machine learning model to determine the grade.

In some embodiments, the machine learning model adopts neural network and/or deep learning for training, and takes all kinds of images of malocclusion classes and required correction time as training data. As a result, the classification rules of the image features corresponding to all kinds of malocclusion classes may be established, and the grade is calculated based on the classification rules.

In some embodiments, taking four types of malocclusion classes A1 to A3, B as an example. The classification rules record the relationship between “all kinds of occlusion states, upper/lower teeth profile, face profile” and “malocclusion classes A1 to A3, B”. For example, what kind of occlusion state, upper/lower teeth profile, face profile may make the correction type to be malocclusion class A1 or malocclusion class B.

In the step S15, the credibility computing module 34 is configured to determine a credibility of the malocclusion class based on the occlusion grade, the lower teeth profile grade, the upper teeth profile grade and the side face profile grade.

In some embodiments, the occlusion grade includes multiple occlusion credibility of the occlusion state belonging in multiple malocclusion classes. The lower teeth profile grade includes multiple lower teeth profile credibility of the arrangement of the lower teeth image belonging in the multiple malocclusion classes. The upper teeth profile grade includes multiple lower teeth profile credibility of the arrangement of the upper teeth image belonging in the multiple malocclusion classes. The side face profile grade includes multiple side face profile credibility of the side face profile belonging in the multiple malocclusion classes.

Further, the credibility computing module 34 may be configured to determine that the user belongs in multiple credibility of the multiple malocclusion classes based on the multiple occlusion credibility, the multiple lower teeth profile credibility, the multiple lower teeth profile credibility, and the multiple side face profile credibility.

As a result, the user may evaluate the proper orthodontic treatment and time according to the credibility of each malocclusion class.

In some embodiments, the multiple credibility may be defined by a grade range. Two terminal values of the grade range respectively indicate the highest credibility and a lowest credibility.

For example, the grade range may be 0% to 100%. 0% is the lowest credibility. 100% is the highest credibility.

In some other embodiments, the grade range may be one (“1”) to five (“5”). One is the lowest credibility. Five is the highest credibility.

In some other embodiments, the grade range may be “A” to “E”. “E” is the lowest credibility. “A” is the highest credibility.

In some embodiments, the credibility computing module 34 is configured to execute an average computation to the multiple occlusion credibility, the multiple lower teeth profile credibility, the multiple lower teeth profile credibility, and the multiple side face profile credibility to compute the multiple credibility.

In some embodiments, the average computation is a weighted average computation. The multiple occlusion credibility are at a highest weighting and the multiple side face profile credibility are at a lowest weighting.

In some embodiments, the weighted proportion of the credibility is distributed as 35% to the occlusion credibility, 27.5% to the lower teeth profile credibility, 27.5% to the upper teeth profile credibility and 10% to the side face profile credibility, here is not intended to be limiting.

In some embodiments, the credibility computing module 34 may be configured to execute an extremum computation to the multiple occlusion credibility, the multiple lower teeth profile credibility, the multiple lower teeth profile credibility, and the multiple side face profile credibility to select the maximum or minimum as the credibility.

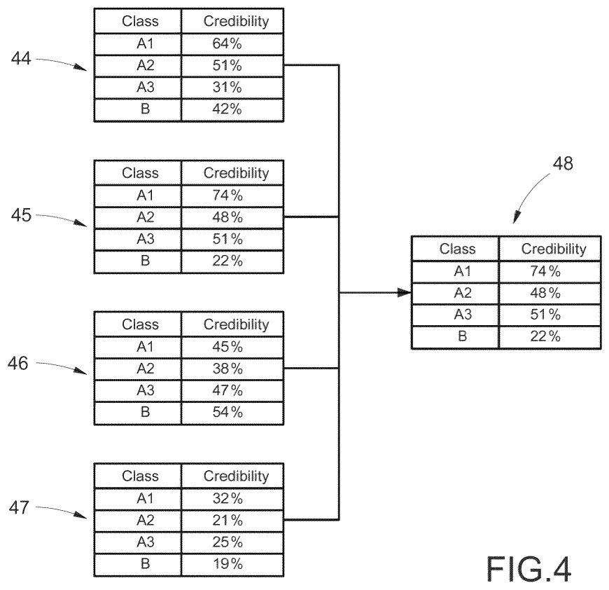

Pleaser refer to FIG. 4, which is a schematic diagram of calculating credibility of the disclosure in accordance with some embodiments.

In some embodiments, the disclosure may be used to determine four types of malocclusion classes, which are the malocclusion class A1 (such as, the minor correction with correction time less than six months), the malocclusion class A2 (such as, the minor correction with correction time between six to twelve months), the malocclusion class A3 (such as, the minor correction with correction time between twelve to eighteen months) and the malocclusion class B (such as, the normal correction needs tooth extraction or the other surgery).

In some embodiments, the occlusion grade 44, the lower teeth profile grade 45, the upper teeth profile grade 46 and the side face profile grade 47 are calculated based on the image of corresponding angles and respectively include the credibility of four types of malocclusion classes.

The credibility computing module 34 may be configured to perform maximum computation to the occlusion grade 44, the lower teeth profile grade 45, the upper teeth profile grade 46 and the side face profile grade 47 to determine the credibility 48.

The credibility of the malocclusion classes A1, A2, A3, B in the credibility 48 are all selected from the maximum in the grades 44 to 47.

Referring back to FIG. 5, in the step S16, the computer device 1 is configured to output the malocclusion class.

In some embodiments, the computer device 1 may be configured to output all malocclusion classes and the corresponding credibility.

In some embodiments, the computer device 1 may be configured to solely output the malocclusion class with the highest credibility.

As a result, the disclosure may be used to provide the user to recognize the malocclusion class by personal computer device in home.

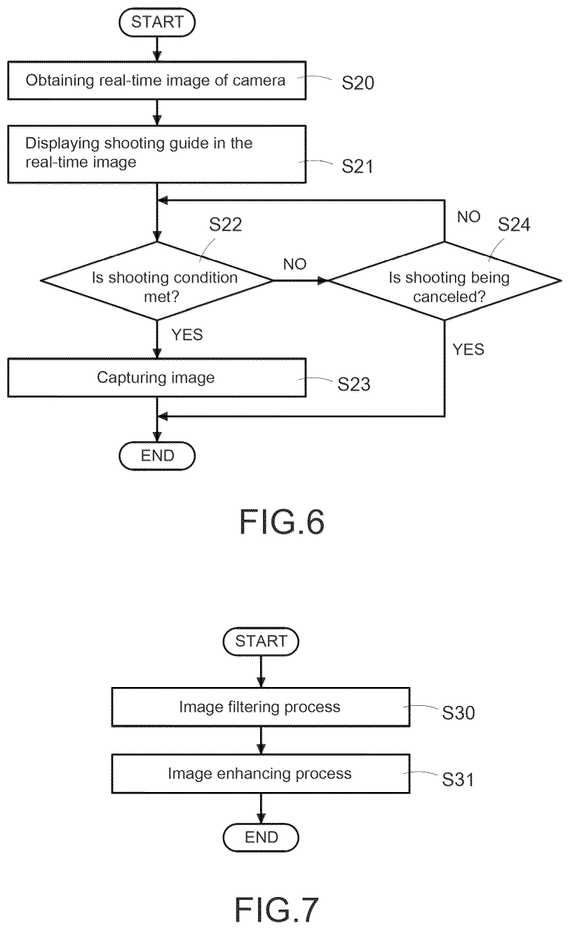

Please refer to FIG. 5 and FIG. 6, FIG. 6 is a partial flowchart of the method of the disclosure in accordance with some embodiments.

The step S10 of the method in the embodiment is repeatedly executing the step S20 to step S24 to respectively obtain the user's occlusion image, lower teeth profile image, upper teeth profile image, and side face image.

In the step S20, the guiding and positioning module 35 may be configured to continuously obtain a real-time image through a camera 11.

In the step S21, the guiding and positioning module 35 may be configured to display the real-time image in real-time through the display of the HMI 14, and display a shooting guide.

In some embodiments, the shooting guide may be pattern or text. When the shooting guide is pattern (for example, teeth profile pattern or aiming pattern), the user may determine whether the teeth image is located at the appropriate shooting position. When the shooting guide is text, the guiding and positioning module 35 may be configured to detect the position of the teeth image in the real-time image in real-time, and guide the user to move the teeth position in the screen through text (such as, “moving upward”, “moving downward”, etc.).

In the step S22, the guiding and positioning module 35 may be configured to determine whether the preset shooting condition is met.

In some embodiments, the shooting condition is that the shooting button is pressed by the user.

In some embodiments, the shooting condition is that the position of the teeth image is consistent with the shooting guide.

In some embodiments, the shooting condition is that the position of the teeth image is consistent with the shooting guide and the shooting button is pressed by the user.

If the shooting condition is met, the step S23 is executed. If the shooting condition is not met, the step S24 is executed.

In the step S23, the guiding and positioning module 35 may be configured to capture the real-time image at the very time as one of the occlusion image, the lower teeth profile image, the upper teeth profile image, or the side face image.

In the step S24, the guiding and positioning module 35 may be configured to determine whether the shooting is canceled. For example, the user terminates the program or cancels the shooting manually.

If the condition of canceling the shooting is met, the execution of the method is terminated. If the shooting is not canceled, the step S22 is re-executed.

In some embodiments, the guiding and positioning module 35 may be configured to switch between the occlusion shooting mode, lower teeth profile shooting mode, upper teeth profile shooting mode and side face shooting mode, and execute the step S20 to S24 under each mode to obtain corresponding image.

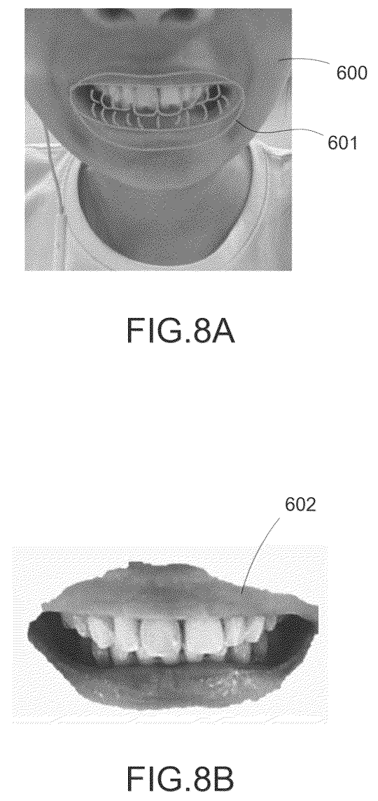

Please refer to FIG. 8A, which is a schematic diagram of the occlusion image of the disclosure in accordance with some embodiments.

The guiding and positioning module 35 may be configured to display the real-time image 600 in real-time through the display under the occlusion shooting mode, and overlay the occlusion pattern 601 on the real-time image 600 to be the shooting guide.

As a result, when the user moves, the real-time image 600 is renewed in real-time, and the user may change the relative position between the teeth image in the real-time image 600 and the occlusion pattern 601 instantaneously to determine whether the appropriate shooting position is reached.

Further, the guiding and positioning module 35 may be configured to capture the real-time image at the very time for the occlusion image, when image of the occlusion portion (such as, the lip and exposed upper/lower teeth) in the real-time image 600 is overlapped with the occlusion pattern 601.

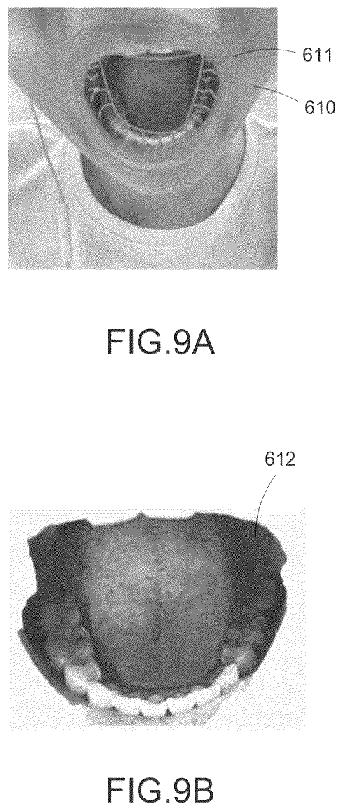

Please refer to FIG. 9A, which is a schematic diagram of the lower teeth profile image of the disclosure in accordance with some embodiments.

The guiding and positioning module 35 may be configured to display the real-time image 610 in real-time through the display under the lower teeth profile shooting mode, and overlay the lower teeth profile pattern 611 on the real-time image 610 to be the shooting guide.

Further, the guiding and positioning module 35 may be configured to capture the real-time image at the very time for the lower teeth profile image, when image of the lower teeth portion in the real-time image 610 is overlapped with the lower teeth profile pattern 611.

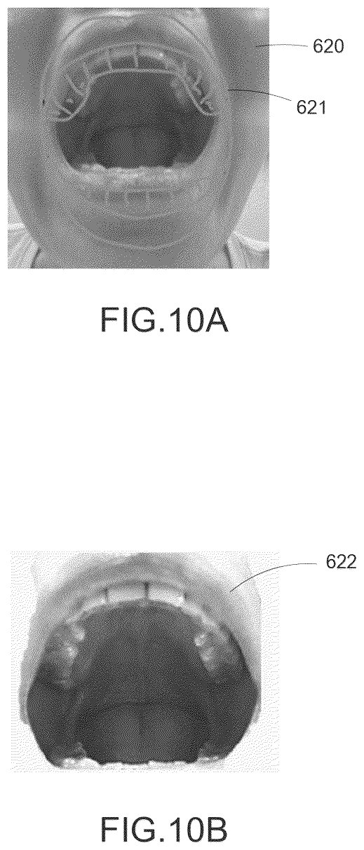

Please refer to FIG. 10A, which is a schematic diagram of the upper teeth profile image of the disclosure in accordance with some embodiments.

The guiding and positioning module 35 may be configured to display the real-time image 620 in real-time through the display under the upper teeth profile shooting mode, and overlay the upper teeth profile pattern 621 on the real-time image 620 to be the shooting guide.

Further, the guiding and positioning module 35 may be configured to capture the real-time image at the very time for the upper teeth profile image, when image of the upper teeth portion in the real-time image 620 is overlapped with the upper teeth profile pattern 621.

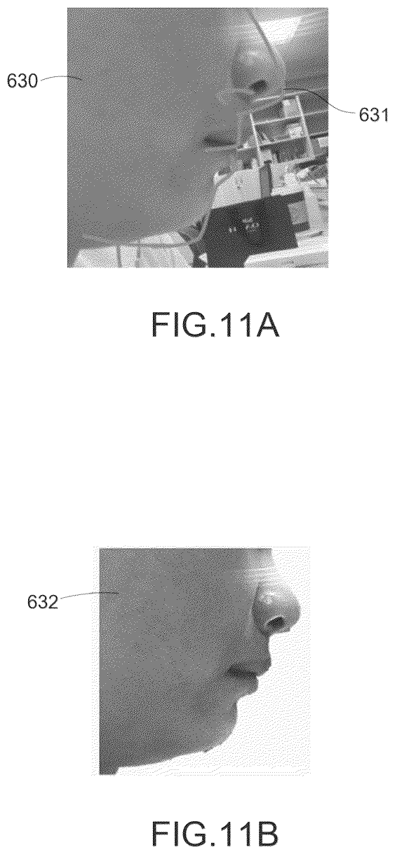

Please refer to FIG. 11A, which is a schematic diagram of the side face image of the disclosure in accordance with some embodiments.

The guiding and positioning module 35 may be configured to display the real-time image 630 in real-time through the display under the side face shooting mode, and overlay the side face pattern 631 on the real-time image 630 to be the shooting guide.

Further, the guiding and positioning module 35 may be configured to capture the real-time image at the very time for the side face image, when image of the side face portion (such as, the range indicated by the side face profile) in the real-time image 630 is overlapped with the side face pattern 631.

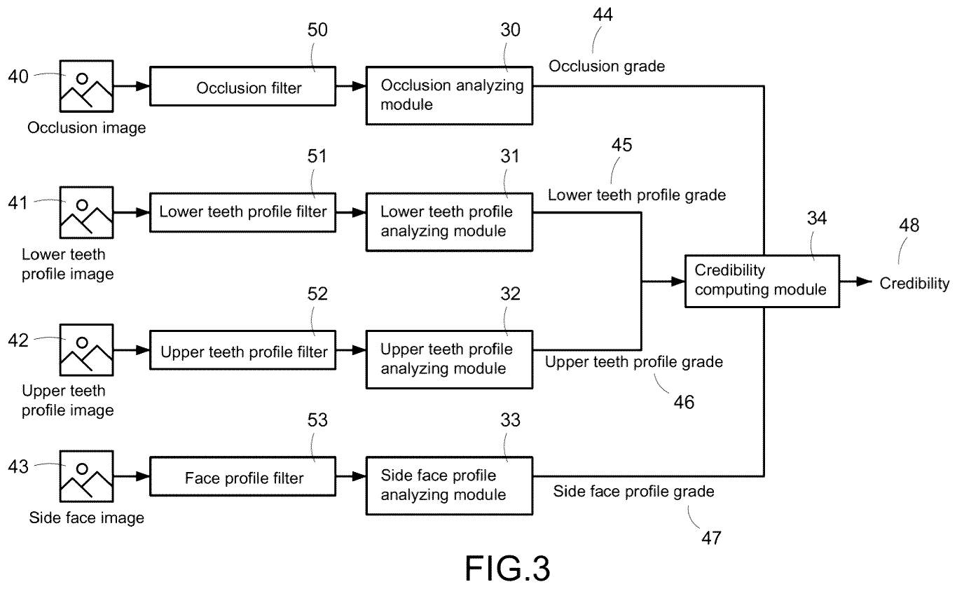

Please refer to FIG. 3, which is a schematic diagram of the data stream of the disclosure in accordance with some embodiments.

In some embodiments, in order to increase liability of the grade, an occlusion filter 50, a lower teeth profile filter 51, an upper teeth profile filter 52 and a face profile filter 53 are further applied. The filters 50 to 53 are similar to the modules 30 to 36, which may be a software module, a hardware module or a combination of software module and hardware module, here is not intended to be limiting.

In some embodiments, the occlusion image 40 is input to the occlusion filter 50. The occlusion filter 50 is configured to execute the image filtering process to the occlusion image 40 to filter unrequired image portion to obtain the required image of occlusion portion.

The image of occlusion portion is input to the occlusion analyzing module 30 for calculating the occlusion grade 44.

Further, the lower teeth profile image 41 is input to the lower teeth profile filter 51. The lower teeth profile filter 51 is configured to execute the image filtering process to the lower teeth profile image 41 to filter unrequired image portion to obtain the required image of lower teeth portion.

The image of lower teeth portion is input to the lower teeth profile analyzing module 31 for calculating the lower teeth profile grade 45.

Further, the upper teeth profile image 42 is input to the upper teeth profile filter 52. The upper teeth profile filter 52 is configured to execute the image filtering process to the upper teeth profile image 42 to filter unrequired image portion to obtain the required image of upper teeth portion.

The image of upper teeth portion is input to the upper teeth profile analyzing module 32 for calculating the upper teeth profile grade 46.

Further, the side face image 43 is input to the face profile filter 53. The face profile filter 53 is configured to execute the image filtering process to the side face image 43 to filter unrequired image portion to obtain the required image of side face portion.

The image of side face portion is input to the side face profile analyzing module 33 for calculating the side face profile grade 47.

At the end, the credibility computing module 34 may be configured to calculate the highest credibility 48 based on the occlusion grade 44, the lower teeth profile grade 45, the upper teeth profile grade 46 and the side face profile grade 47.

Please refer to FIG. 5 and FIG. 7, FIG. 7 is a partial flowchart of the method of the disclosure in accordance with some embodiments. The method of the embodiment may include the steps S30 to S31 between the step S10 and the steps S11 to S14. The steps S30 to S31 may be independent steps, and there may be no sequential relation or dependent relation between the steps.

In the step S30, the image processing module 36 is configured to execute the image filtering process to the image to filter unrequired image portion to obtain image of designated portion, such as occlusion portion, lower teeth portion, upper teeth portion and side face portion.

In some embodiments, the image processing module 36 may include the filters 50 to 53, and is configured to respectively execute the image filtering process to images with different angles though the filters 50 to 53.

Please refer to FIG. 8A to FIG. 11B, FIG. 8B is a schematic diagram of the image of the occlusion portion of the disclosure in accordance with some embodiments, FIG. 9B is a schematic diagram of the image of the lower teeth portion of the disclosure in accordance with some embodiments, FIG. 10B is a schematic diagram of the image of the upper teeth portion of the disclosure in accordance with some embodiments, and FIG. 11B is a schematic diagram of the image of the side face portion of the disclosure in accordance with some embodiments.

The occlusion filter 50 is configured to execute the image filtering process to the occlusion image (such as, the captured real-time image 600) to obtain the image 602 of occlusion portion.

The lower teeth profile filter 51 is configured to execute the image filtering process to the lower teeth profile image (such as, the captured real-time image 610) to obtain the image 612 of lower teeth portion.

The upper teeth profile filter 52 is configured to execute the image filtering process to the upper teeth profile image (such as, the captured real-time image 620) to obtain the image 622 of upper teeth portion.

The face profile filter 53 is configured to execute the image filtering process to the side face image (such as, the captured real-time image 630) to obtain the image 632 of side face portion.

In the step S31, the image processing module 36 is configured to execute the image enhancing process to the image before filtering or after filtering to obtain image of emphasized portion.

In some embodiments, the image enhancing process may include a grey scale process, a half-tone process, an edge-enhancing process, a contrast-enhancing process, and/or a noise-canceling process, etc., here is not intended to be limiting.

In some embodiments, the image processing module 36 may be configured to execute the image enhancing process to the occlusion image to obtain enhanced (or emphasized) image of the occlusion portion, execute the image enhancing process to the lower teeth profile image to obtain enhanced (or emphasized) image of the lower teeth portion, execute the image enhancing process to the upper teeth profile image to obtain enhanced (or emphasized) image of the upper teeth portion, and execute the image enhancing process to the side face image to obtain enhanced (or emphasized) image of the side face portion.

The disclosure may reduce the interference in the image through the image filtering process and image enhancing process to increase the liability of the grade.

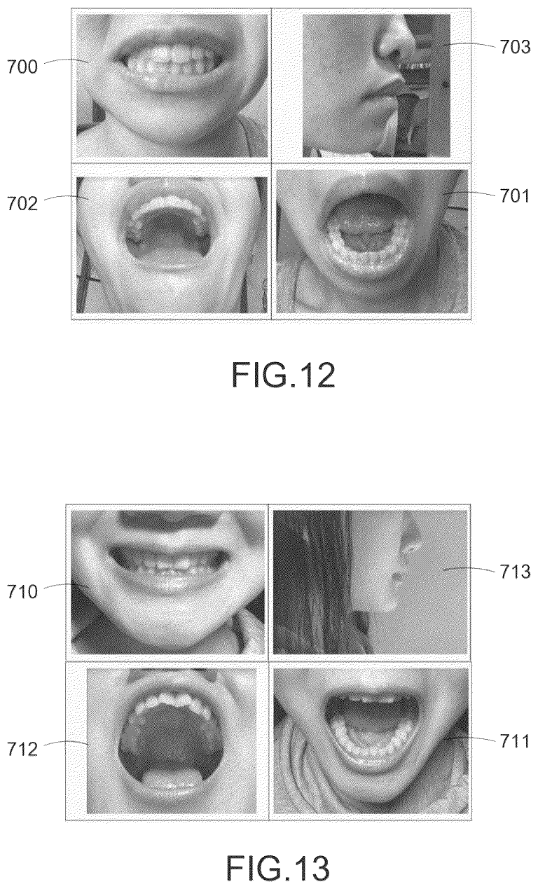

Please refer to FIG. 12, which is an image schematic diagram of the malocclusion class A1 of the disclosure in accordance with some embodiments.

The occlusion image 700, the lower teeth profile image 701, the upper teeth profile image 702, and the side face image 703 are the oral images in multi-angles of the patient with the malocclusion class A1.

In some embodiments, the definition of the malocclusion class A1 is that the occlusion is normal, and the level of irregular dentition or crevice between teeth is minor and that is concentrated on the incisor portion or part of the dentition.

Please refer to FIG. 13, which is an image schematic diagram of the malocclusion class A2 of the disclosure in accordance with some embodiments.

The occlusion image 710, the lower teeth profile image 711, the upper teeth profile image 712, and the side face image 713 are the oral images in multi-angles of the patient with the malocclusion class A2.

In some embodiments, the definition of the malocclusion class A2 is that the level of irregular dentition or crevice between teeth is minor, and all dentition in the mouth need to be moved to complete the treatment.

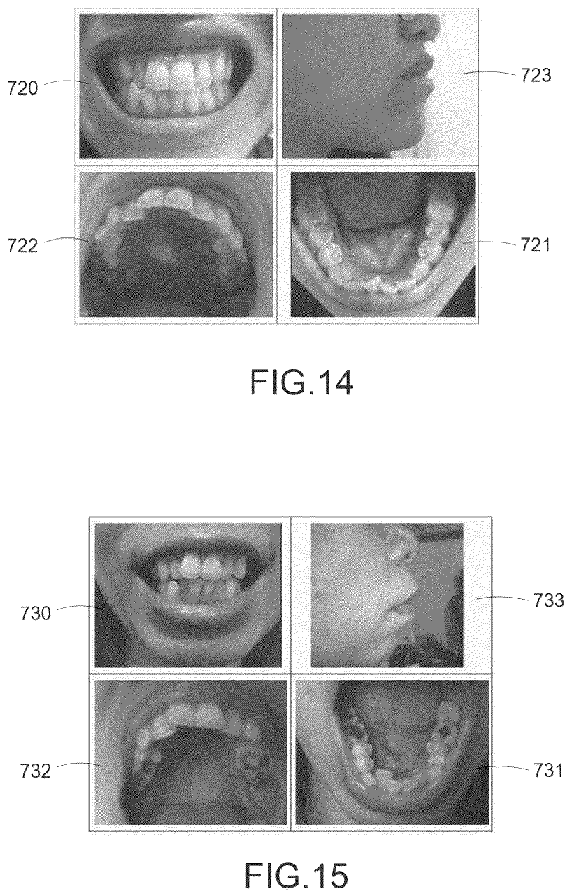

Please refer to FIG. 14, which is an image schematic diagram of the malocclusion class A3 of the disclosure in accordance with some embodiments.

The occlusion image 720, the lower teeth profile image 721, the upper teeth profile image 722, and the side face image 723 are the oral images in multi-angles of the patient with the malocclusion class A3.

In some embodiments, the definition of the malocclusion class A3 is that the level of irregular dentition or crevice between teeth is moderate, some parts are false occlusion or protruding jaw, and all dentition in the mouth need more space and time to complete the treatment.

Please refer to FIG. 15, which is an image schematic diagram of the malocclusion class B of the disclosure in accordance with some embodiments.

The occlusion image 730, the lower teeth profile image 731, the upper teeth profile image 732, and the side face image 733 are the oral images in multi-angles of the patient with the malocclusion class B.

In some embodiments, the definition of the malocclusion class B is that the dentition is squeezed or the occlusion problem is severe, many false occlusion, tooth extraction is required for many teeth (such as, extracting four premolars) for additional space to align the dentition and improve severe occlusion in Angle's level 2 and level 3.

While this disclosure has been described by means of specific embodiments, numerous modifications and variations may be made thereto by those skilled in the art without departing from the scope and spirit of this disclosure set forth in the claims.

What is claimed is:

1. A method of automatically recognizing a malocclusion class, the method comprising:
a) obtaining, by a computer device, an occlusion image, a lower teeth profile image, an upper teeth profile image, and a side face image;
b) computing an occlusion grade based on an occlusion state of a teeth image in the occlusion image;
c) computing a lower teeth profile grade based on an arrangement of a lower teeth image in the lower teeth profile image;
d) computing an upper teeth profile grade based on an arrangement of an upper teeth image in the lower teeth profile image;
e) computing a side face profile grade based on a side face profile in the side face image;
f) determining at least one credibility of at least one malocclusion class based on the occlusion grade, the lower teeth profile grade, the upper teeth profile grade, and the side face profile grade; and
g) outputting, by the computer device, the malocclusion class with a highest credibility.

2. The method according to claim 1, wherein the a) further comprises:
a1) continuously obtaining a real-time image through a camera;
a2) displaying the real-time image in real-time, and displaying a shooting guide; and
a3) capturing the real-time image as the occlusion image, the lower teeth profile image, the upper teeth profile image, or the side face image, when a shooting condition is met.

3. The method according to claim 2, wherein the a2) comprises:
a21) displaying the real-time image in real-time under an occlusion shooting mode, and overlaying an occlusion pattern with the real-time image to be the shooting guide;
a22) displaying the real-time image in real-time under a lower teeth profile shooting mode, and overlaying a lower teeth profile pattern with the real-time image to be the shooting guide;
a23) displaying the real-time image in real-time under an upper teeth profile shooting mode, and overlaying an upper teeth profile pattern with the real-time image to be the shooting guide; and
a24) displaying the real-time image in real-time under a side face shooting mode, and overlaying a side face pattern with the real-time image to be the shooting guide.

4. The method according to claim 3, wherein the a3) comprises:
a31) capturing the real-time image as the occlusion image, when image of an occlusion portion in the real-time image under the occlusion shooting mode is overlapped with the occlusion pattern;
a32) capturing the real-time image as the lower teeth profile image, when image of a lower teeth portion in the real-time image under the lower teeth profile shooting mode is overlapped with the lower teeth profile pattern;
a33) capturing the real-time image as the upper teeth profile image, when image of an upper teeth portion in the real-time image under the upper teeth profile shooting mode is overlapped with the upper teeth profile pattern; and
a34) capturing the real-time image as the side face image, when image of a side face portion in the real-time image under the side face shooting mode is overlapped with the side face pattern.

5. The method according to claim 1, wherein after the a) and before the b) to the g), the method further comprises:
h1) executing an image filtering process to the occlusion image to obtain image of an occlusion portion;
h2) executing the image filtering process to the lower teeth profile image to obtain image of a lower teeth portion;
h3) executing the image filtering process to the upper teeth profile image to obtain image of an upper teeth portion; and
h4) executing the image filtering process to the side face image to obtain image of a side face portion.

6. The method according to claim 1, wherein after the a) and before the b) to the g), the method further comprises:
i1) executing an image enhancing process to the occlusion image to obtain enhanced image of an occlusion portion;
i2) executing the image enhancing process to the lower teeth profile image to obtain enhanced image of a lower teeth portion;
i3) executing the image enhancing process to the upper teeth profile image to obtain enhanced image of an upper teeth portion; and
i4) executing the image enhancing process to the side face image to obtain enhanced image of a side face portion.

7. The method according to claim 1, wherein the occlusion grade comprises multiple occlusion credibility of the occlusion state belonging in multiple malocclusion classes;
the lower teeth profile grade comprises multiple lower teeth profile credibility of the arrangement of the lower teeth image belonging in the multiple malocclusion classes;
the upper teeth profile grade comprises multiple lower teeth profile credibility of the arrangement of the upper teeth image belonging in the multiple malocclusion classes;
the side face profile grade comprises multiple side face profile credibility of the side face profile belonging in the multiple malocclusion classes; and
the f) further comprises:
determining that the user belongs in multiple credibility of the multiple malocclusion classes based on the multiple occlusion credibility, the multiple lower teeth profile credibility, the multiple lower teeth profile credibility, and the multiple side face profile credibility.

8. The method according to claim 7, wherein the multiple credibility is defined by a grade range, and two terminal values of the grade range respectively indicate the highest credibility and a lowest credibility.

9. The method according to claim 7, wherein the f) further comprises:
executing an average computation to the multiple occlusion credibility, the multiple lower teeth profile credibility, the multiple lower teeth profile credibility, and the multiple side face profile credibility to compute the multiple credibility.

10. The method according to claim 9, wherein the f) further comprises:
executing a weighted average computation, wherein the multiple occlusion credibility are at a highest weighting and the multiple side face profile credibility are at a lowest weighting.

11. A system of automatically recognizing a malocclusion class, the system comprising:
an occlusion analyzing module, configured to compute an occlusion grade based on an occlusion state of a teeth image in an occlusion image of a user;
a lower teeth profile analyzing module, configured to compute a lower teeth profile grade based on an arrangement of a lower teeth image in a lower teeth profile image of the user;
an upper teeth profile analyzing module, configured to compute an upper teeth profile grade based on an arrangement of an upper teeth image in an upper teeth profile image of the user;
a side face profile analyzing module, configured to compute a side face profile grade based on a side face profile in a side face image of the user; and
a credibility computing module, configured to determine a credibility of at least one malocclusion class based on the occlusion grade, the lower teeth profile grade, the upper teeth profile grade, and the side face profile grade, and output the malocclusion class with a highest credibility.

12. The system according to claim 11, further comprising:
a guiding and positioning module, configured to continuously obtain a real-time image through a camera, display the real-time image in real-time and a shooting guide through a display, and capture the real-time image as the occlusion image, the lower teeth profile image, the upper teeth profile image, or the side face image when a shooting condition is met.

13. The system according to claim 12, wherein the guiding and positioning module is configured to display the real-time image in real-time under an occlusion shooting mode, and overlay an occlusion pattern with the real-time image to be the shooting guide;
the guiding and positioning module is configured to display the real-time image in real-time under a lower teeth profile shooting mode, and overlay a lower teeth profile pattern with the real-time image to be the shooting guide;
the guiding and positioning module is configured to display the real-time image in real-time under an upper teeth profile shooting mode, and overlay an upper teeth profile pattern with the real-time image to be the shooting guide; and
the guiding and positioning module is configured to display the real-time image in real-time under a side face shooting mode, and overlay a side face pattern with the real-time image to be the shooting guide.

14. The system according to claim 13, wherein the guiding and positioning module is configured to capture the real-time image as the occlusion image, when image of an occlusion portion in the real-time image under the occlusion shooting mode is overlapped with the occlusion pattern;
the guiding and positioning module is configured to capture the real-time image as the lower teeth profile image, when image of a lower teeth portion in the real-time image under the lower teeth profile shooting mode is overlapped with the lower teeth profile pattern;
the guiding and positioning module is configured to capture the real-time image as the upper teeth profile image, when image of an upper teeth portion in the real-time image under the upper teeth profile shooting mode is overlapped with the upper teeth profile pattern; and
the guiding and positioning module is configured to capture the real-time image as the side face image, when image of a side face portion in the real-time image under the side face shooting mode is overlapped with the side face pattern.

15. The system according to claim 11, further comprising:
an image processing module, configured to execute an image filtering process to the occlusion image to obtain image of an occlusion portion, execute the image filtering process to the lower teeth profile image to obtain image of a lower teeth portion, execute the image filtering process to the upper teeth profile image to obtain image of an upper teeth portion, and execute the image filtering process to the side face image to obtain image of a side face portion.

16. The system according to claim 11, further comprising:
an image processing module, configured to execute an image enhancing process to the occlusion image to obtain enhanced image of an occlusion portion, execute the image enhancing process to the lower teeth profile image to obtain enhanced image of a lower teeth portion, execute the image enhancing process to the upper teeth profile image to obtain enhanced image of an upper teeth portion, and execute the image enhancing process to the side face image to obtain enhanced image of a side face portion.

17. The system according to claim 11, wherein the occlusion grade comprises multiple occlusion credibility of the occlusion state belonging in multiple malocclusion classes;
the lower teeth profile grade comprises multiple lower teeth profile credibility of the arrangement of the lower teeth image belonging in the multiple malocclusion classes;
the upper teeth profile grade comprises multiple lower teeth profile credibility of the arrangement of the upper teeth image belonging in the multiple malocclusion classes;
the side face profile grade comprises multiple side face profile credibility of the side face profile belonging in the multiple malocclusion classes; and
the credibility computing module is configured to determine that the user belongs in multiple credibility of the multiple malocclusion classes based on the multiple occlusion credibility, the multiple lower teeth profile credibility, the multiple lower teeth profile credibility, and the multiple side face profile credibility.

18. The system according to claim 17, wherein the multiple credibility is defined by a grade range, and two terminal values of the grade range respectively indicate the highest credibility and a lowest credibility;
the credibility computing module is configured to execute a weighted average computation to the multiple occlusion credibility, the multiple lower teeth profile credibility, the multiple lower teeth profile credibility, and the multiple side face profile credibility to compute the multiple credibility; and
the multiple occlusion credibility are at a highest weighting and the multiple side face profile credibility are at a lowest weighting.

19. The system according to claim 11, further comprising:
a server, configured to be connected to a computer device comprising the camera and the display through an Internet, obtain the occlusion image, the lower teeth profile image, the upper teeth profile image and the side face image, and output the malocclusion class with the highest credibility to the computer device;
wherein the server comprises the occlusion analyzing module, the lower teeth profile analyzing module, the upper teeth profile analyzing module, the side face profile analyzing module, and the credibility computing module.

20. A computer program of automatically recognizing a malocclusion class, configured to be stored in a computer device, and implement the method according to claim 1 after being executed by the computer device.
